(19) United States
(12) Patent Application Publication
(10) Pub. No.: US 2022/0265742 A1
(43) Pub. Date: Aug. 25, 2022

(54) NANO-DISPERSION METHODS FOR CANNABIS EXTRACT

(71) Applicant: University of Vermont and State Agricultural College, Burlington, VT (US)

(72) Inventors: Monique MCHENRY, Burlington, VT (US); William L. CATS-BARIL, Burlington, VT (US); Kalev FREEMAN, Burlington, VT (US); Mingruo GUO, Burlington, VT (US)

(21) Appl. No.: 17/625,134

(22) PCT Filed: Jul. 6, 2020
(86) PCT No.: PCT/US20/40874

§ 371 Date: Jan. 6, 2022

Related U.S. Application Data
(60) Provisional application No. 62/871,113, filed on Jul. 6, 2019.

(51) Int. Cl. A61K 36/185; A61K 9/107; A61K 47/46

(52) U.S. Cl. CPC A61K 36/185; A61K 9/1075; A61K 47/46; A61K 2236/37; A61K 2236/35

(57) ABSTRACT

The present disclosure provides techniques for producing whey protein nano-emulsions from cannabis extracts. An emulsion prepared using an embodiment of a method of the invention can be subject to further processing with which to convert the emulsion (or a component thereof) into one or more consumables.

### BACKGROUND OF THE INVENTION

Cannabis is a flowering plant that may be classified by its intoxicating and non-intoxicating constituents. Plants producing an abundance of intoxicating constituents are often referred to as marijuana, whereas those with non-intoxicating constituents are referred to as hemp.

Oils extractable from hemp include non-intoxicating cannabinoids, flavonoids, and terpenes. Each of these non-intoxicating oils have therapeutic potential.

Hemp oils and extracts are available as tinctures and in various commercial products. However, suitable methods for formulating hemp oils for consistent dosing and delivery of its active ingredients remain unavailable. The inability to efficiently produce consistent cannabis extracts impacts both clinical research, potential therapeutics, as well as the commercialization of industrial hemp products.

### SUMMARY OF THE INVENTION

Aspects of the present disclosure relate techniques for producing whey protein nano-emulsions from cannabis extracts. In at least some embodiments of the invention, the full spectrum of bioactive compounds is retained and can be easily scaled for industrial use.

An aspect of the present disclosure relates to a whey protein nano-dispersion emulsification method including: producing a bio extract from cannabis; producing an organic phase oil nano-emulsion by combining the bio extract with an oil at a ratio from 1:1 to 1:3 (v/v); adding the organic phase oil nano-emulsion to an aqueous phase including at least one of a whey protein isolate (WPI) and a whey protein concentrate (WPC); and producing a course emulsion using the organic phase and aqueous phase. In at least some embodiments of the invention, a means of producing the bio extract includes supercritical carbon dioxide bio-extraction. In at least some embodiments of the invention, a means of producing the bio extract includes an organic solvent based extraction. In at least some embodiments of the invention, the oil includes a food grade oil. In at least some embodiments of the invention, the food grade oil includes at least one of olive oil and sesame oil. In at least some embodiments of the invention, the method further includes decarboxylating the organic phase oil nano-emulsion prior to adding the organic phase oil nano-emulsion to the aqueous phase. In at least some embodiments of the invention, the decarboxylating is performed in a heated environment. In at least some embodiments of the invention, the heated environment includes a water bath. In at least some embodiments of the invention, the heated environment includes an oven. In at least some embodiments of the invention, the decarboxylated organic phase oil nano-emulsion is at least about 60% decarboxylated. In at least some embodiments of the invention, the decarboxylated organic phase oil nano-emulsion is between about 60% and about 95% decarboxylated. In at least some embodiments of the invention, the bio extract is about 0.1% to about 20% of the course emulsion. In at least some embodiments of the invention, the bio extract is about 0.1% to about 5% of the course emulsion. In at least some embodiments of the invention, the adding the organic phase oil nano-emulsion is performed using an organic phase oil nano-emulsion to aqueous phase ratio selected from the group consisting of 1:1, 1:2, 1:3, 1:4, 1:5, 1:6, 1:7, 1:8, and 1:9. In at least some embodiments of the invention, a means of producing the course emulsion includes high-speed blending. In at least some embodiments of the invention, the method further includes homogenizing the course emulsion to produce a homogenized course emulsion. In at least some embodiments of the invention, the homogenizing includes sonication. In at least some embodiments of the invention, the sonication includes ultrasonic sonication. In at least some embodiments of the invention, the method further includes producing a powder from the homogenized course emulsion. In at least some embodiments of the invention, the producing of the powder includes removing the aqueous phase from the homogenized course emulsion. In at least some embodiments of the invention, the removing includes freeze-drying. In at least some embodiments of the invention, the removing includes spray-drying. In at least some embodiments of the invention, the removing includes heated drying. In at least some embodiments of the invention, the method further includes preparing the powder for administration to a subject, by: pressing the powder into a tablet; adding the powder to a capsule; or dissolving the powder in a liquid. In at least some embodiments of the invention, the liquid includes water. In at least some embodiments of the invention, the cannabis includes hemp. In at least some embodiments of the invention, the cannabis includes an inflorescence of the hemp, optionally a dried inflorescence of the hemp. In at least some embodiments of the invention, the bio extract includes at least one of cannabinoids, flavonoids, and terpenoids.

Another aspect of the present disclosure relates to a whey protein nano-dispersion emulsification method, including: producing a bio extract from cannabis; producing an organic phase oil nano-emulsion by blending the bio extract without a carrier oil; adding the organic phase oil nano-emulsion to an aqueous phase including at least one of a whey protein isolate (WPI) and a whey protein concentrate (WPC); and producing a course emulsion using the organic phase and aqueous phase. In at least some embodiments of the invention, the producing of the bio extract includes a supercritical carbon dioxide bio-extraction. In at least some embodiments of the invention, the producing of the bio extract includes an organic solvent based extraction. In at least some embodiments of the invention, the method further includes decarboxylating the organic phase oil nano-emulsion prior to adding the organic phase oil nano-emulsion to the aqueous phase. In at least some embodiments of the invention, the decarboxylating is performed in a heated environment. In at least some embodiments of the invention, the heated environment includes a water bath. In at least some embodiments of the invention, the heated environment includes an oven. In at least some embodiments of the invention, the decarboxylated organic phase oil nano-emulsion is at least about 60% decarboxylated. In at least some embodiments of the invention, the decarboxylated organic phase oil nano-emulsion is between about 60% and about 95% decarboxylated. In at least some embodiments of the invention, the bio extract is about 0.1% to about 20% of the course emulsion. In at least some embodiments of the invention, the bio extract is about 0.1% to about 5% of the course emulsion. In at least some embodiments of the invention, the adding is performed using an organic phase oil nano-emulsion to aqueous phase ratio selected from the group consisting of 1:1, 1:2, 1:3, 1:4, 1:5, 1:6, 1:7, 1:8, and 1:9. In at least some embodiments of the invention, a means of producing the course emulsion includes high-speed blending. In at least some embodiments of the invention, the method further includes homogenizing the course emulsion to produce a homogenized course emulsion. In at least some embodiments of the invention, the homogenizing includes sonication. In at least some embodiments of the invention, the sonication includes ultrasonic sonication. In at least some embodiments of the invention, the method further includes producing a powder from the homogenized course emulsion. In at least some embodiments of the invention, the producing of the powder includes removing the aqueous phase from the homogenized course emulsion. In at least some embodiments of the invention, the removing includes freeze-drying. In at least some embodiments of the invention, the removing includes spray-drying. In at least some embodiments of the invention, the removing includes heated drying. In at least some embodiments of the invention, the method further includes preparing the powder for administration to a subject, by: pressing the powder into a tablet; adding the powder to a capsule; or dissolving the powder in a liquid. In at least some embodiments of the invention, the liquid includes water. In at least some embodiments of the invention, the cannabis includes hemp. In at least some embodiments of the invention, the cannabis includes an inflorescence of hemp, optionally a dried inflorescence of hemp. In at least some embodiments of the invention, the bio extract includes at least one of cannabinoids, flavonoids, and terpenoids.

### DETAILED DESCRIPTION

The present disclosure provides techniques for producing whey protein nano-emulsions from cannabis extracts. In at least some embodiments of the invention, a bio extract may be produced from cannabis. The bio extract may be combined with an oil [non-limiting examples of which are at ratios from 1:1 to 1:3 (v/v)] to produce an organic phase oil nano-emulsion. The organic phase oil nano-emulsion may be added to an aqueous phase including whey protein isolate (WPI) and/or whey protein concentrate (WPC). A course emulsion may be produced using the organic phase and aqueous phase. In at least some embodiments of the invention, the bio extract is not combined with an oil and in such embodiments, the organic phase oil nano-emulsion (added to the aqueous phase) may simply be the bio extract. The course emulsion may be subject to various processes to convert the course emulsion (or a component thereof) into one or more consumables.

As used herein the terms hemp and cannabis refers to the genus Cannabis, which contains all species in the genus, including but not limited to C. sativa, C. indica, and C. ruderalis. All three species are of the family Cannabaceae. The term “cannabis” as used herein is intended to encompass plants of the genus Cannabis and/or the family Cannabaceae. A cannabis plant may also be referred to in the art as hemp, a term generally used in reference to non-drug varieties of cannabis. Cannabis has long been used for hemp fiber, hemp seeds and their oils, hemp leaves for use as vegetables and as juice, for medicinal purposes, and as a recreational drug.

Over 450 chemical constituents are known to be present in cannabis plants. The constituents include over 80 identified terpeno-phenolic compounds, which are referred to as cannabinoids and are the bioactive chemical constituents that result in mental and physical effects when consumed. All of the types of cannabinoids are derived from a common precursor compound, cannabigerol (CBG). The cannabis plant also contains a variety of terpenoids.

The two cannabinoids usually produced in greatest abundance are cannabidiol (CBD) and Δ⁹-tetrahydrocannabinol (THC), which is also referred to as: “tetrahydrocannabinol”. Of the two, only THC is psychoactive. The ratio of THC/CBD in a plant is genetically determined and plants with a high ratio may be referred to as “drug” plants and plants with a low ratio may be referred to as “non-drug” plants. Other of the cannabinoid compounds include, but are not limited to: cannabigerol (CBG), cannabichromene (CBC), cannabigerivarin (CBGV); tetrahydrocannabivarin (THCV); cannabidivarin (CBDV), and cannabichromevarin (CBCV).

The following is the chemical formula of THC:

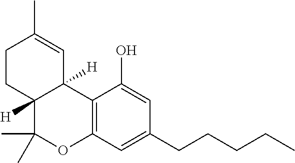 [SMILES: CCCCCc1cc(O)c2c(c1)OC(C)(C)[C@@H]1CCC(C)=C[C@@H]21]

The following is the chemical formula of CBD:

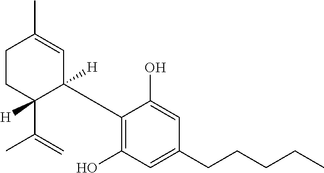 [SMILES: C=C(C)[C@@H]1CCC(C)=C[C@H]1Cc1c(O)cc(CCCCC)cc1O]

Starting material for use in a method of the invention to produce whey protein nano-emulsions from cannabis extracts may include any suitable cannabis starting material as known in the art. Non-limiting examples include raw cannabis plant material, which means material originating from any region of the cannabis plant. Non-limiting examples of regions of cannabis plants are: stems, seeds, flowers, leaves, pistils, colas, calyxs, trichomes, buds (including dormant buds, axillary buds, and terminal buds), petiole, rachis, bract, and roots. The plant material used in methods of the invention may be fresh, though in certain embodiments the material is dried, frozen, or in another preserved state. In some embodiments of the invention, the starting, raw material has not been exposed to pesticides. In certain embodiments of the invention, the raw material is obtained from organically grown cannabis.

It should be understood that the composition of a whey protein nano-emulsion prepared from cannabis extracts using an embodiment of a method of the invention will depend on the starting materials. For example, though not intended to be limiting, the composition of the final concentrated cannabinoid product (on a weight basis, volume basis, etc.), including the concentration of cannabinoids and other chemicals present therein, depends at least in part on the composition of the starting material used in the preparation.

### Bio Extracts

Embodiments of the present disclosure may use bio extracts produced from cannabis. As used herein, “cannabis” refers to a genus of flowering plants in the family Cannabaceae. At least some embodiments of the invention use bio extracts produced from hemp. While cannabis may include significant amounts of intoxicating and/or psychoactive constituents, hemp may have significantly lower concentrations of intoxicating constituents as compared to non-intoxicating constituents. Non-limiting examples of non-intoxicating constituents of hemp include certain cannabinoids, flavonoids, and terpenes. As used herein, a “flavonoid” refers to a chemical compound having a 15-carbon skeletal structure, which may include two phenyl rings and a heterocyclic ring. The chemical structure of a flavonoid may be abbreviated as C₆-C₃-C₆. A flavonoid may be a ketone-containing compound. As used herein, the term “terpene” refers to one or more of a large group of volatile unsaturated hydrocarbons that are present in essential oils of plants. Terpene structures may be based on a cyclic molecule having the formula C₁₀H₁₆. One or more terpenes in cannabis may provide distinctive flavors and/or scents, and different terpenes may be used to promote different effects in humans, from relaxation and stress-relief to focus and acuity.

In at least some embodiments of the invention, a bio extract may be produced from cannabis (and more particularly hemp in at least some embodiments) using supercritical carbon dioxide bio-extraction. Supercritical carbon dioxide bio-extraction is a form of supercritical fluid extraction, which separates one component (i.e., an extractant) from another (i.e., a matrix) using a supercritical fluid (e.g., carbon dioxide) as an extracting solvent. In at least some embodiments of the invention, the matrix may be a solid matrix. In at least some embodiments of the invention, the solid matrix may be dried inflorescence of cannabis (and, in some instances, more particularly hemp). As used herein, “inflorescence” refers to a complete flower head of a plant, include the stems, stalks, bracts, and flowers.

Generally, a supercritical carbon dioxide bio-extraction system may include components such as: a carbon dioxide supply, a pump, an oven comprising a heating means (a non-limiting example of which is a heating coil), an extraction cell, and a metering valve. Carbon dioxide, from the carbon dioxide supply, may be pumped to the heating means where the carbon dioxide is heated to supercritical conditions. The heated carbon dioxide may then be passed into the extraction cell. In the extraction cell, the heated carbon dioxide may diffuse into a solid matrix and dissolve cannabinoids, flavonoids, terpenes, and/or other non-intoxicating constituents of cannabis (and more particularly hemp). The dissolved non-intoxicating constituents may then be passed from the extraction cell into a lower pressure area, where the non-intoxicating constituents may settle out of the heated carbon dioxide, and may be released from the metering valve as the aforementioned bio extract.

In at least some embodiments, organic solvent-based extraction may be performed to produce a bio extract. Art-known organic solvent-based extraction techniques may be used. A non-limiting list of organic solvents that may be used in organic solvent-based cannabinoid extraction include butane, propane, and ethanol. The low boiling point of these solvents allows extractors to remove them without risking evaporating heat-sensitive cannabinoids or terpenes. Ethanol is well suited for large-scale extractions. Butane and propane extraction technology can produce a product with lighter color and more of a terpene-rich smell.

While the foregoing describes certain extraction techniques that may be used to produce a bio extract from cannabis and/or hemp, the present disclosure is not limited thereto. There are various known techniques for producing bio extracts from plants, and it is within the knowledge of one skilled in the art to apply such other known techniques in accordance with various embodiments of the present disclosure.

In at least some embodiments of the invention, the bio extract, produced from cannabis or hemp, may be a resin containing one or more cannabinoids, one or more flavonoids, and/or one or more terpenes. A bio extract of the present disclosure may be considered full spectrum in that it may contain all (or nearly all) of the non-intoxicating constituents of the cannabis (or hemp) dried inflorescence or plant material from which it was extracted in the extraction cell.

### Oils

Embodiments of the present disclosure may use one or more oils. In at least some embodiments of the invention, one or more of the one or more oils may be a food-grade oil. Non-limiting examples of food-grade oils are: olive oil, extra virgin olive oil, sesame oil, coconut oil, almond oil, avocado oil, canola oil, coconut oil, corn oil, cottonseed oil, grapeseed oil, hazelnut oil, palm oil, peanut oil, palm seed oil, pumpkin oil, safflower oil, soy oil, sunflower oil, vegetable oil, and walnut oil. A skilled artisan will be able to determine other suitable art-known food-grade oils that are suitable for use in certain embodiments of the invention.

### Whey Protein Concentrate and Whey Protein Isolate

Embodiments of the present disclosure may use whey protein concentrate (WPC) and/or whey protein isolate (WPI). Whey protein is a mixture of proteins isolated from whey, such as α-lactalbumin, β-lactoglobulin, serum albumin, and immunoglobulins.

As used herein, the term “whey protein concentrate” (WPC) refers to a mixture comprising at least 30% whey protein. In at least some embodiments of the invention, a WPC may have at least 80% whey protein, and more particularly between 80% to 85% whey protein. In certain embodiments of the invention, a WPC comprises at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, or 89% whey protein.

As used herein, “whey protein isolate” or “WPI” refers to a mixture including at least 90% whey protein. In at least some embodiments of the invention, a WPI may include between 90% to 92% whey protein. In certain embodiments of the invention, WPI comprises at least 90%, 91%, 92%, 93%, 94%, 95%, 96%, 97%, 98%, or 99% whey protein.

### Method of Producing a Whey Protein Nano-Emulsion

In at least some embodiments of the invention, a method of producing a whey protein nano-dispersion emulsification may begin with producing a bio extract from cannabis as detailed herein above.

In at least some embodiments of the invention, an organic phase oil nano-emulsion may be produced by combining the bio extract with one or more oils. In at least some embodiments of the invention, the bio extract may be combined with one or more oils at a ratio of 1:1 (v/v). In at least some embodiments of the invention, the bio extract may be combined with one or more oils at a ratio of 1:2 (v/v). In at least some embodiments of the invention, the bio extract may be combined with one or more oils at a ratio of 1:3 (v/v). In at least some embodiments of the invention, an organic phase oil nano-emulsion may be generated by melting the bio extract with the oil.

In at least some embodiments of the invention, the organic phase oil nano-emulsion may be decarboxylated (i.e., undergo decarboxylation). Decarboxylation is a chemical reaction that remove carboxyl groups and, as a result, releases carbon dioxide. In at least some embodiments of the invention, decarboxylation may remove one or more carboxyl groups present in the bio extract of the organic phase oil nano-emulsion. In at least some embodiments of the invention, as a result of decarboxylation, the bio extract may be at least 50% decarboxylated, at least 55% decarboxylated, at least 60% decarboxylated, at least 65% decarboxylated, at least 70% decarboxylated, at least 75% decarboxylated, at least 80% decarboxylated, at least 85% decarboxylated, at least 90% decarboxylated, at least 95% decarboxylated, or more. In some embodiments of the invention, the bio extract is at least 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% decarboxylated.

In at least some embodiments of the invention, decarboxylation of the organic phase oil nano-emulsion ion may be performed in a heated environment, such as a water bath or oven for example. The bio extract (or combined bio extract and oil depending on the embodiment) may be maintained at various temperatures during decarboxylation of the bio extract. Example temperatures include, but are not limited to at least 100° C., at least 105° C., at least 110° C., at least 115° C., at least 120° C., at least 125° C., at least 130° C., at least 135° C., at least 140° C., at least 145° C., at least 150° C., or more. In some embodiments of the invention the bio extract is maintained for a period of time during decarboxylation at a temperature of at least: 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., or 160° C.

Moreover, the bio extract (or combined bio extract and oil depending on the embodiment) may undergo decarboxylation for various durations of time. Example durations of time include, but are not limited to at least 50 min, at least 60 min, at least 70 min, at least 80 min, at least 90 min, at least 100 min, at least 110 min, at least 120 min, at least 130 min, or more. In some embodiments of the invention, the time of the decarboxylation is at least 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 852, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 minutes long.

In at least some embodiments of the invention, decarboxylation of the bio extract may include heating a container, holding the bio extract, on a hot plate without stirring or rotation. Decarboxylation may occurs as the bio extract is melted.

The organic phase oil nano-emulsion (either without undergoing decarboxylation or after undergoing decarboxylation) may be added to an aqueous phase include WPC and/or WPI. In at least some embodiments of the invention, the bio extract may not be combined with an oil(s). In such embodiments, the organic phase oil nano-emulsion (added to the aqueous phase) may simply be the bio extract. It is thus worth noting that, when an oil(s) is combined with the bio extract to produce the organic phase oil nano-emulsion, the oil functions as a dilutant, rather than a base or carrier. In certain embodiments of the invention, the organic phase oil nano-emulsion may be added to the aqueous phase in various ratios. In at least some embodiments of the invention, an organic phase oil nano-emulsion to aqueous phase ratio may be 1:1, 1:2, 1:3, 1:4, 1:5, 1:6, 1:7, 1:8, 1:9, or more.

A coarse emulsion may be produced from the combined organic phase oil nano-emulsion and aqueous phase. In at least some embodiments of the invention, the coarse emulsion may be produced by subjecting the combined organic phase oil nano-emulsion and aqueous phase to a high-speed blender (e.g., an Ultra-Turrax T25 high-speed blender). The bio extract may be present in the course emulsion in various concentrations. In at least some embodiments of the invention, the bio extract may be 0.1% to 20% of the course emulsion. In at least some embodiments of the invention, the bio extract may be 0.1% to 5% of the course emulsion. In certain embodiments of the invention, the bio extract may be at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% pf the course emulsion.

In at least some embodiments, the coarse emulsion may be homogenized to produce a homogenized course emulsion. The coarse emulsion may be homogenized in various manners. In at least some embodiments of the invention, the coarse emulsion may be homogenized via sonication, which is the act of applying sound energy to agitate particles in a sample. In at least some embodiments of the invention, the coarse emulsion may be homogenized via ultrasonic sonication, which may use frequencies greater than 20 kHz. In at least some embodiments of the invention, a Vibra-Cell may be used to homogenize the coarse emulsion.

### Consumables

In at least some embodiments of the invention, a powder may be produced from a homogenized course emulsion. Production of the powder, in at least some embodiments of the invention, involves removing the aqueous phase from the homogenized course emulsion. Freeze-drying, spray-drying, heated drying, or some other known drying technique may be used to remove the aqueous phase from the homogenized course emulsion.

The powder may be prepared for administration to a subject. In at least some embodiments of the invention, the powder may be pressed into a tablet. In at least some embodiments of the invention, the powder may be added to a capsule. In at least some embodiments of the invention, the powder may be dissolved in a liquid, a non-limiting example of which is water.

The powder (or the pre-powder emulsion) may be incorporated into various food compositions, a non-limiting list of which includes dairy products, ice creams, sauces, soups, desserts, confectionary products, bakery products, salad dressings, and pet foods. The powder (or the pre-powder emulsion) may additionally by incorporated in beverages, a non-limiting list of which includes bottled-water based drinks, energy drinks, milk drinks, and tea beverages. The powder (or pre-powder emulsion) may additionally be incorporated in cosmetic products. It will be appreciated that the emulsions produced according to the present disclosure (and powders produced therefrom) have various applications, and that the foregoing uses are non-limiting examples.

### EXAMPLES

Experiments were carried out in which bio extract was combined with a food grade oil either prior to or after the No extract underwent decarboxylation. The decarboxylation of the bio extract was performed at different temperatures and for different durations of time. Below is a table summarizing certain experiments.

TABLE 1
Experimental conditions in certain embodiments of the invention

| | | When Bio | | | |
|---|---|---|---|---|---|
| | Type and | Extract | | | |
| Amount of Bio | Amount | Combined | | Duration | % |
| Extract | of Oil | with Oil | Temperature | of Time | Decarboxylation |
| 13.2 g | Olive oil - 30 g | Before heating | 120° C. | 60 min | 100 |
| (decarboxylated) | | | | | |
| 21.10 g | Olive oil - 22 g | Before | 120° C. | 60 min | 50 |
| | | decarboxylation | | | |
| 15 g | Sesame oil - 15 g | Before | 145° C. | 120 min | 67 |
| | | decarboxylation | | | |
| 15 g | None | — | 145° C. | 120 min | 70 |
| 15 g | Olive oil - 15 g | Before | 150° C. | 120 min | 70 |
| | | decarboxylation | | | |
| 15 g | O oil - 15 g | After | 150° C. | 120 min | 87 |
| | | decarboxylation | | | |

As illustrated in the above table, decarboxylation up to 87% is achievable in accordance with embodiments of the invention. Moreover, it should be noted that significant decarboxylation may be observed even when an oil is combined with the bio extract after the bio extract is decarboxylated.

Although the present disclosure has been particularly described in conjunction with specific embodiments, it is evident that many alternatives, modifications, and variations will be apparent to those skilled in the art in light of the foregoing description. It is therefore contemplated that the appended claims will embrace any such alternatives, modifications, and variations as falling within the true spirit and scope of the present disclosure.

1. A whey protein nano-dispersion emulsification method, comprising:
producing a bio extract from cannabis;
producing an organic phase oil nano-emulsion by combining the bio extract with an oil at a ratio from 1:1 to 1:3 (v/v);
adding the organic phase oil nano-emulsion to an aqueous phase comprising at least one of a whey protein isolate (WPI) and a whey protein concentrate (WPC); and
producing a course emulsion using the organic phase and aqueous phase.

2. The method of claim 1, wherein a means of producing the bio extract comprises one or both of a supercritical carbon dioxide bio-extraction and an organic solvent based extraction.

3. (canceled)

4. The method of claim 1, wherein the oil comprises a food grade oil.

5. (canceled)

6. The method of claim 1, further comprising decarboxylating the organic phase oil nano-emulsion prior to adding the organic phase oil nano-emulsion to the aqueous phase.

7-9. (canceled)

10. The method of claim 6, wherein the decarboxylated organic phase oil nano-emulsion is at least about 60% decarboxylated.

11. (canceled)

12. The method of claim 1, wherein the bio extract is about 0.1% to about 20% of the course emulsion.

13. (canceled)

14. The method of claim 1, wherein the adding the organic phase oil nano-emulsion is performed using an organic phase oil nano-emulsion to aqueous phase ratio selected from the group consisting of 1:1, 1:2, 1:3, 1:4, 1:5, 1:6, 1:7, 1:8, and 1:9.

15. (canceled)

16. The method of claim 1, further comprising homogenizing the course emulsion to produce a homogenized course emulsion.

17-18. (canceled)

19. The method of claim 16, further comprising producing a powder from the homogenized course emulsion.

20-25. (canceled)

26. The method of claim 1, wherein the cannabis comprises hemp.

27. (canceled)

28. The method of claim 1, wherein the bio extract comprises at least one of cannabinoids, flavonoids, and terpenoids.

29. A whey protein nano-dispersion emulsification method, comprising:
producing a bio extract from cannabis;
producing an organic phase oil nano-emulsion by blending the bio extract without a carrier oil;
adding the organic phase oil nano-emulsion to an aqueous phase comprising at least one of a whey protein isolate (WPI) and a whey protein concentrate (WPC); and
producing a course emulsion using the organic phase and aqueous phase.

30. The method of claim 29, wherein the producing of the bio extract comprises one or both of a supercritical carbon dioxide bio-extraction and an organic solvent based extraction.

31. (canceled)

32. The method of claim 29, further comprising decarboxylating the organic phase oil nano-emulsion prior to adding the organic phase oil nano-emulsion to the aqueous phase.

33-35. (canceled)

36. The method of claim 32, wherein the decarboxylated organic phase oil nano-emulsion is at least about 60% decarboxylated.

37. (canceled)

38. The method of claim 29, wherein the bio extract is about 0.1% to about 20% of the course emulsion.

39. (canceled)

40. The method of claim 29, wherein the adding is performed using an organic phase oil nano-emulsion to aqueous phase ratio selected from the group consisting of 1:1, 1:2, 1:3, 1:4, 1:5, 1:6, 1:7, 1:8, and 1:9.

41. (canceled)

42. The method of claim 29, further comprising homogenizing the course emulsion to produce a homogenized course emulsion.

43-44. (canceled)

45. The method of claim 42, further comprising producing a powder from the homogenized course emulsion.

46-51. (canceled)

52. The method of claim 29, wherein the cannabis comprises hemp.

53-54. (canceled)
